(19) United States
(12) Patent Application Publication
(10) Pub. No.: US 2023/0349005 A1
(43) Pub. Date: Nov. 2, 2023

(54) EXOGENOUS BIOMOLECULAR TRACING METHOD AND SYSTEM THEREOF FOR AQUATIC CREATURES AND PRODUCTS

(71) Applicant: National Taiwan Ocean University, Keelung (TW)

(72) Inventors: TE-HUA HSU, Keelung (TW); HUNG-TAI LEE, Keelung (TW); HONG-YI GONG, Keelung (TW); CHANG-WEN HUANG, Keelung (TW)

(21) Appl. No.: 17/848,118

(22) Filed: Jun. 23, 2022

(30) Foreign Application Priority Data
May 2, 2022 (TW) 111116589

(51) Int. Cl. C12Q 1/6897; C12N 15/10

(52) U.S. Cl. CPC C12Q 1/6897; C12N 15/1065; C12Q 1/6844

(57) ABSTRACT

An exogenous biomolecular tracing method includes: extracting a nucleotide marker from a marker source organism; combining a basic material with the nucleotide marker to form a nucleotide marked material; exogenously combining an aquatic creature or an aquatic product to form an exogenously-marked aquatic creature or an exogenously-marked aquatic product; and identifying a DNA sample from the exogenously-marked aquatic creature or the exogenously-marked aquatic product with at least one primer pair in an exogenous biomolecular tracing procedure to obtain an exogenously-marked identification result.

### SEQUENCE LISTING

This sequence listing is created on 08/30/2022 with the file name “P22006TWTOU_ST25_SF22_0003 UP_0830” and file size 33407 bytes; the entire contents of which are hereby incorporated by reference.

### BACKGROUND OF THE INVENTION

### 1. Field of the Invention

The present invention relates to an exogenous biomolecular tracing method and system thereof for aquatic creatures and products. Particularly, the present invention relates to the exogenous biomolecular tracing method and system thereof with nucleotide markers for aquatic creatures and products.

### 2. Description of the Related Art

By way of example, Taiwanese Patent Application Publication No. TW-200911131, entitled “Feed composition for aquatic organisms,” discloses a feed composition including a protein, an additional protein, a lipid, an additional lipid, a carbohydrate and water which are formed particles ranging between 1 to 100 micrometers.

The sources of protein are provided from fish meat or high-quality fish meat, the lipid is provided from fish oils, the additional lipid is provided from cholesterol, vegetable oils or lecithin, with the lecithin is selected from egg yolk or soybeans. The additional lipid includes fish hydrosylate, hill, cuttlefishes or extracts of yeasts.

The feed composition further includes at least one mineral substance and at least one vitamin, with the mineral substance including trace elements and calcium salts, the calcium salt selected from calcium carbonate or calcium phosphate and the vitamins including vitamin A, vitamin B complex, vitamin C and vitamin E.

Further, another Taiwanese Patent Publication No. TW-I656835, entitled “Breeding and baiting system and method thereof for fry of aquatic creatures,” discloses a breeding and baiting system and method thereof, including a breeding tank, a water inlet, a water supply conduit and a water outlet.

The breeding tank includes an inner space portion and an opening portion, with disposing the water inlet and the water outlet at a first predetermined position and a second predetermined position of the inner space portion, with connecting the water supply conduit with the inner space portion or the water inlet to continuously or intermittently supply a predetermined water jet flow.

In breeding and baiting operation, the predetermined water jet flow is applied to drive at least one bait material to thereby form a driven dynamic bait so that a plurality of fry can chase the driven dynamic bait in the breeding tank.

Further, another Taiwanese Patent Publication No. TW-I660669, entitled “Artificial sausage-casing type feed, baiting device and method thereof for aquatic creatures,” discloses a baiting device, including an artificial sausage-type feed, a baiting support frame, a floating device and an operation member.

The artificial sausage-casing-type feed has a rope member, at least one insert member and at least one casing in which to form an inner space to enclose a bait filling. An end of the rope member connects with a connection end of insert member while an insert end of insert member inserts into the filled casing to form the artificial sausage-casing-type feed.

The baiting support frame has a fixing bar on which to connect the rope member of the artificial sausage-casing-type feed. The floating device further connects with the fixing bar of the baiting support frame for providing a floating force thereon.

The operating member has a trolling rope member to connect the floating device to form the baiting device which is provided under a water surface. In a baiting operation the operation member can be manually or automatically controlled to troll the baiting support frame to provide the artificial sausage-type feed to at least one aquatic creature for chasing, catching and eating it.

Further, another Taiwanese Patent Publication No. TW-I695679, entitled “Auxiliary light system and method thereof for baiting aquatic creatures,” discloses an auxiliary light system, including a baiting light device or a plurality of baiting light devices.

The baiting light device can be operated to project a movable light beam or a plurality of movable light beams to form a dynamic light spot or a plurality of dynamic light spots under water surface. In a baiting operation, aquatic creatures can chase, catch and bite the dynamic light spots for auxiliary baiting operation.

Further, another Taiwanese Patent Publication No. TW-1706723, entitled “Stress relief formula for fry or adult of cephalopods of aquatic creatures,” discloses a stress relief formula, including at least one cephalopod extract and a water solution.

The cephalopod extract is extracted from at least one cephalopod and the water solution is added to the cephalopod extract to produce a cephalopod stress relief formula. The cephalopod stress relief formula can be applied to add to a cultural water body for protecting fry or adult of cephalopods therein.

Further, another UK Patent Application Publication No. GB-2395414, entitled “Gel feed for fish,” discloses a gel feed. The gel feed is an animal feed composition (or aquatic animal composition) which includes at least one dry powdered diet for forming the gel feed.

When the dry powdered diet selectively mixed with wet feed and/or with water, the dry powdered diet can be formed as a gel. The animal feed composition further includes an algae material performed as a binder therein.

Furthermore, the animal feed composition may optionally include a liquid component.

Further, another Chinese Patent Application Publication No. CN-110964845, entitled “Method capable of tracing maize mixed pollination hybrid seed sources and InDel molecular marker,” discloses a method of tracing maize mixed pollination hybrid seed sources and an InDel molecular marker.

The method comprises the steps: (1) extracting DNA of all male parents, female parents and mixed pollination hybrid seed leaves to be detected; (2) screening molecular markers capable of establishing a fingerprint spectrum by utilizing the DNA of the male parent and the female parent.

The method further comprises the steps: (3) performing PCR amplification on the DNA template in step (1) by using the screened molecular markers, and recording genotypes of the male parents, the female parents and the mixed pollination hybrid seeds to be detected; (4) deriving all possible hybrid genotypes according to the obtained genotypes of the male parents and the female parents.

The method further comprises the steps: (5) comparing the genotypes of the mixed pollination hybrid seeds to be detected obtained in step (3) with the genotypes of all the hybrid seeds derived in step (4), and recording the number of sites with the same genotype.

The method further comprises the step: If a certain mixed pollination hybrid seed to be detected and a certain derived hybrid seed have the maximum site number of the same genotype, the parent of the derived hybrid seed is derived as the parent source of the mixed pollination hybrid seeds to be detected.

Further, another PCT (Patent Cooperation Treaty) Patent Application Publication No. WO-2011/108062, entitled “Method for labeling and identifying fishery fish and shellfish by mitochondrial DNA variable region base sequence,” discloses a method for labeling and identifying fishery fish and shellfish by mitochondrial DNA variable region base sequence.

The method for labeling and identification of fishery fish and shellfish is a simple and accurate method for labeling and identification of fish and shellfish, particularly fishery fish and shellfish derived from a specific production area, or superior fishery eggs and fry having one or a plurality of superior characterizing features, or a specific type of fishery fish and shellfish.

The method for labeling and identification of fishery fish and shellfish is a method for labeling and determining or tracking the profile of a single fishery fish or shellfish, an individual school of fishery fish or shellfish, a portion thereof, or the processed product thereof, by using the D loop base sequence of mitochondrial DNA. The method is also provided to label fishery fish and shellfish, particularly fertilized eggs, juvenile fish, or fingerlings.

However, there is a need of improving the conventional method for labeling and identification of fishery fish and shellfish performed as an identification of unique nucleotide marker for aquatic creatures and products. The above-mentioned patents and patent application publications are incorporated herein by reference for purposes including, but not limited to, indicating the background of the present invention and illustrating the situation of the art.

### SUMMARY OF THE INVENTION

The primary objective of this invention is to provide an exogenous biomolecular tracing method and system thereof for aquatic creatures and products thereof. A nucleotide marker is extracted from a marker source organism, with further combining a basic material with the nucleotide marker to form a nucleotide marker-contained material. An aquatic creature or an aquatic product is applied to exogenously combine with the nucleotide marker-contained material to form an exogenously-marked aquatic creature or an exogenously-marked aquatic product. In tracing operation, at least one primer pair is provided to identify a DNA sample received from the exogenously-marked aquatic creature or the exogenously-marked aquatic product in an exogenous biomolecular tracing procedure to obtain an exogenously-marked identification result. Advantageously, the exogenous biomolecular tracing method and system of the present invention is successful in tracing original aquatic creatures and products thereof.

The exogenous biomolecular tracing system in accordance with an aspect of the present invention includes:
a nucleotide marker extracted from a marker source organism with an extraction device;
a nucleotide marker-contained material formed from combining a basic material with the nucleotide marker;
an exogenously-marked aquatic creature or an exogenously-marked aquatic product formed from exogenously combining the nucleotide marker-contained material with an aquatic creature or an aquatic product;
at least one primer pair corresponding to the nucleotide marker; and
an exogenously-marked identification result obtained by identifying a DNA sample received from the exogenously-marked aquatic creature or the exogenously-marked aquatic product with the at least one primer pair in an exogenous biomolecular tracing procedure with a DNA testing device;
wherein the exogenously-marked identification result shows a correct source of original aquatic creature or product or an incorrect source of original aquatic creature or product.

In a separate aspect of the present invention, the nucleotide marker is selected from an insect extract, a plant extract, a microorganism extract or combinations thereof.

In a further separate aspect of the present invention, the basic material is selected from a solution, a powder, a fragment, a solid or combinations thereof.

In yet a further separate aspect of the present invention, the aquatic creature or the aquatic product is selected from fishes, shrimps, shellfishes, algae or combinations thereof.

In yet a further separate aspect of the present invention, the aquatic creature or the aquatic product is selected from edible aquatic creatures, ornamental aquatic creatures, live aquatic creatures, frozen aquatic creatures, aquatic fry or aquatic baits.

The exogenous biomolecular tracing method in accordance with an aspect of the present invention includes:
extracting a nucleotide marker from a marker source organism;
combining a basic material with the nucleotide marker to form a nucleotide marker-contained material;
exogenously combining the nucleotide marker-contained material with an aquatic creature or an aquatic product to form an exogenously-marked aquatic creature or an exogenously-marked aquatic product; and identifying a DNA sample received from the exogenously-marked aquatic creature or the exogenously-marked aquatic product with at least one primer pair in an exogenous biomolecular tracing procedure to obtain an exogenously-marked identification result.

In a separate aspect of the present invention, the nucleotide marker is selected from a DNA extract, a DNA extract liquid, a DNA extract powder, a DNA extract-related material or combinations thereof.

In a further separate aspect of the present invention, exogenously combining the nucleotide marker-contained material with the aquatic creature or the aquatic product includes a feeding process, a dipping process, a painting process, a spraying process or combinations thereof.

In yet a further separate aspect of the present invention, the at least one primer pair is applied to execute a test of polymerase chain reaction (PCR) or loop-mediated isothermal amplification (LAMP).

In yet a further separate aspect of the present invention, the exogenously-marked identification result includes a combination set of codes or code forms for tracing the exogenously-marked aquatic creature or the exogenously-marked aquatic product.

The exogenous biomolecular tracing method in accordance with another aspect of the present invention includes:
extracting a first nucleotide marker from a first marker source organism;
combining a first basic material with the first nucleotide marker to form a first nucleotide marker-contained material;
extracting a second nucleotide marker from a second marker source organism;
combining a second basic material with the second nucleotide marker to form a second nucleotide marker-contained material;
in a first coding stage, exogenously combining the first nucleotide marker-contained material with an aquatic creature or an aquatic product to form a first exogenously-marked aquatic creature or a first exogenously-marked aquatic product;
in a second coding stage, exogenously combining the second nucleotide marker-contained material with the first exogenously-marked aquatic creature or the first exogenously-marked aquatic product to form a second exogenously-marked aquatic creature or a second exogenously-marked aquatic product; and
identifying a DNA sample received from the second exogenously-marked aquatic creature or the second exogenously-marked aquatic product with at least one primer pair in an exogenous biomolecular tracing procedure to obtain an exogenously-marked identification result.

In a separate aspect of the present invention, the first or second nucleotide marker is selected from a DNA extract, a DNA extract liquid, a DNA extract powder, a DNA extract-related material or combinations thereof.

In a further separate aspect of the present invention, exogenously combining the first or second nucleotide marker-contained material with the aquatic creature or the aquatic product includes a feeding process, a dipping process, a painting process, a spraying process or combinations thereof.

In yet a further separate aspect of the present invention, the at least one primer pair is applied to execute a test of polymerase chain reaction (PCR) or loop-mediated isothermal amplification (LAMP).

In yet a further separate aspect of the present invention, the exogenously-marked identification result includes a combination set of codes or code forms for tracing the second exogenously-marked aquatic creature or the second exogenously-marked aquatic product.

Further scope of the applicability of the present invention will become apparent from the detailed description given hereinafter. However, it should be understood that the detailed description and specific examples, while indicating preferred embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a schematic block diagram of an exogenous biomolecular tracing system for aquatic creatures and products in accordance with a preferred embodiment of the present invention.

FIG. 2 is a schematic block diagram of a nucleotide marker applied in an exogenous biomolecular tracing method and system thereof for aquatic creatures and products in accordance with a preferred embodiment of the present invention.

FIG. 3 is a flow chart of an exogenous biomolecular tracing method for aquatic creatures and products in accordance with a preferred embodiment of the present invention.

FIG. 4 is a photograph view of a LAMP reaction applied in the exogenous biomolecular tracing system for aquatic creatures and products in accordance with a preferred embodiment of the present invention.

FIG. 4A is a schematic view of a first code of LAMP reaction generated in the exogenous biomolecular tracing system for aquatic creatures and products in accordance with a first embodiment of the present invention.

FIG. 4B is a schematic view of a second code of LAMP reaction generated in the exogenous biomolecular tracing system for aquatic creatures and products in accordance with a second embodiment of the present invention.

### DETAILED DESCRIPTION OF THE INVENTION

It is noted that an exogenous biomolecular tracing method (or usage) and system (e.g., information system) thereof for aquatic creatures and products thereof in accordance with the preferred embodiment of the present invention can be applicable to various aquatic creature baiting systems, various aquatic fry or creature breeding systems, various aquatic creature display systems, various algae (or microalgae) cultivation systems or various algae display systems, which are not limitative of the present invention.

For instance, an exogenous biomolecular tracing method (or usage) and system (e.g., information system) thereof for aquatic creatures and products thereof in accordance with the preferred embodiment of the present invention can be also applicable to various aquatic creature feeds, e.g., various crustacean feeds (including crabs, crab larvae, shrimps, shrimp larvae, lobsters, lobster larvae, krill, krill larvae etc.), various fish (fry) feeds, various animal feeds or various algae feeds (including algae fish feeds or Taiwan Tilapia feeds), which are not limitative of the present invention.

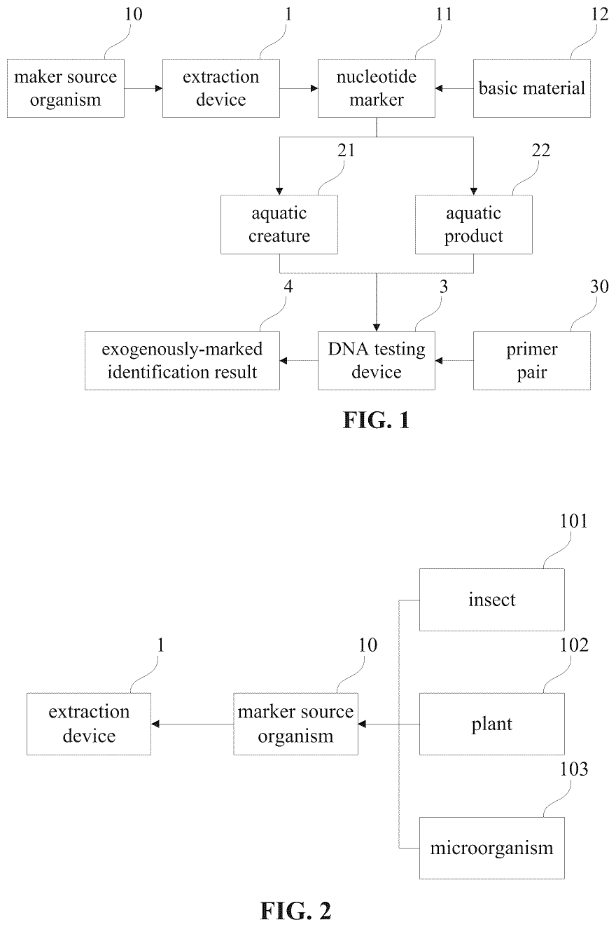

FIG. 1 shows a schematic block diagram of an exogenous biomolecular tracing system for aquatic creatures and products in accordance with a preferred embodiment of the present invention. Referring now to FIG. 1, the exogenous biomolecular tracing system in accordance with the preferred embodiment of the present invention includes a plurality of nucleotide markers 11, a basic material 12, a plurality of aquatic creatures (exogenous aquatic creatures) 21 or aquatic products 22 and at least one primer pair 30.

With continued reference to FIG. 1, by way of example, in another embodiment, the exogenous biomolecular tracing system further includes at least one extraction device 1, at least one DNA testing device 3 and related peripheral devices, e.g. feeding machine, painting machine, spraying machine or other machines.

Still referring to FIG. 1, by way of example, the nucleotide markers 11 can be selectively extracted from various maker source organisms (maker-contained organisms) 10 which can be selected from various positions of organisms (e.g., branches, roots, heads or feet), various parts of organisms (e.g., leaves, seeds, hairs or ova) or various defecation of organisms.

Still referring to FIG. 1, by way of example, the basic material (marker basic material) 12 can be selected from a solution, a powder, a fragment, a solid or combinations thereof and can be also selected from a feed material, a formula feed material, a nutritional supplement material, a medium material, a drinking water, a spray raw material or a medical raw material. Still referring to FIG. 1, by way of example, the aquatic creatures (exogenous aquatic creatures) 21 or aquatic products (exogenous aquatic products) 22 can be selected from fishes, shrimps, shellfishes, algae or combinations thereof and can be also selected from edible aquatic creatures, ornamental aquatic creatures, live aquatic creatures, frozen aquatic creatures, aquatic fry or aquatic baits.

FIG. 2 shows a schematic block diagram of a nucleotide marker applied in an exogenous biomolecular tracing method and system thereof for aquatic creatures and products in accordance with a preferred embodiment of the present invention. Turning now to FIGS. 1 and 2, the maker source organisms 10 (or nucleotide markers 11) can be selected from an insect material (or insect extract) 101, a plant material (or plant extract) 102, a microorganism material (or microorganism extract) 103 or combinations thereof for providing various sets of codes.

Still referring to FIGS. 1 and 2, by way of example, the maker source organisms 10 can be selected from inset materials, plant materials, vegetable materials, seaweed materials, fungus materials, bean materials, fruit materials or other suitable maker source organism materials.

Still referring to FIGS. 1 and 2, by way of example, the inset materials can be selected from Hermetia illucens materials, housefly materials or maggot materials; the plant materials can be selected from Camellia sinensis materials, sweet potato materials, paddy rice materials, wheat materials or Myristica fragrans materials; the vegetable materials can be selected from tomato materials or gracilaria materials; the seaweed materials can be selected from kelp materials or other similar materials.

Still referring to FIGS. 1 and 2, by way of example, the fungus materials can be selected from Lycium chinense materials, Agaricus blazei materials, Agrocybe cylindracea materials, Boletus edulis materials, Coprinus comatus materials, Grifola frondosa (Dickson: Fries) Gray materials, Morchella esculenta (L.) Pers. materials, Termitomyces albuminosus (Berkeley & Broome) Heim materials, Agrocybe cylindracea Murrill materials, Ganoderma tsugae Murrill materials or Cordyceps sinensis materials.

Still referring to FIGS. 1 and 2, by way of example, the bean materials can be selected from green soybean materials, red bean materials, peanut materials, green bean materials, vigna unguiculata materials, kidney bean materials, broad bean materials, pea materials, swordbean materials, pigeon pea or other bean materials.

Still referring to FIGS. 1 and 2, by way of example, the fruit materials can be selected from strawberry materials, orange materials, grape fruit materials, kiwi materials, pitaya materials, guava materials, passion fruit materials, skin materials of papaya, skin materials of hami melon, grape materials, mango materials, banana materials, peach materials, mandarin orange materials, pineapple or other fruit materials.

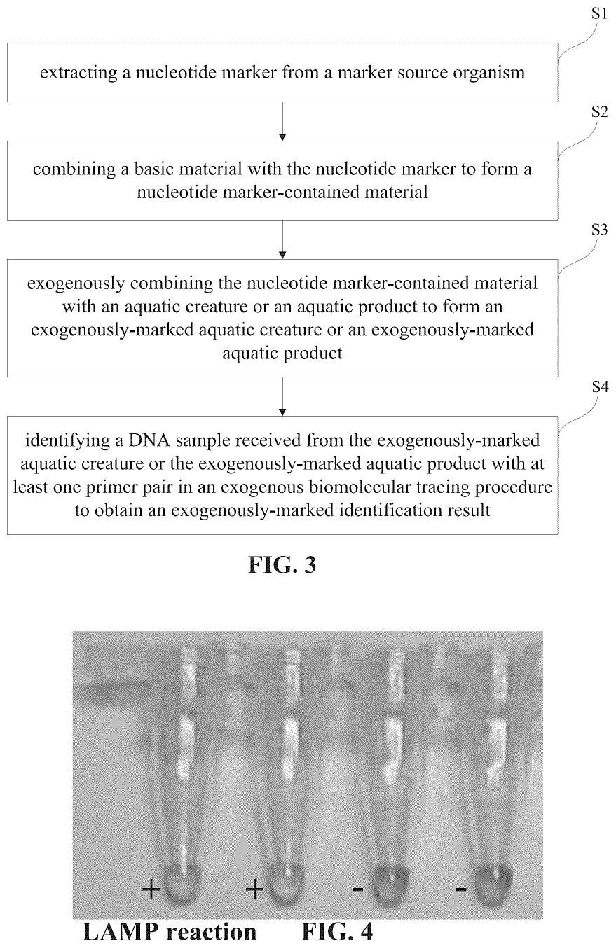

FIG. 3 shows a flow chart of an exogenous biomolecular tracing method for aquatic creatures and products in accordance with a preferred embodiment of the present invention. Turning now to FIGS. 1, 2 and 3, the exogenous biomolecular tracing method in accordance with the preferred embodiment of the present invention includes the step S1: automatically, semi-automatically or manually extracting the nucleotide marker 11 from the marker source organism 10 with the extraction device 1.

With continued reference to FIGS. 1, 2 and 3, by way of example, the nucleotide marker 11 can be selected from a DNA extract, a DNA extract liquid, a DNA extract powder (e.g., freeze-dried powder), a DNA extract-related material or combinations thereof

Still referring to FIGS. 1, 2 and 3, by way of example, the exogenous biomolecular tracing method in accordance with the preferred embodiment of the present invention includes the step S2: automatically, semi-automatically or manually combining the basic material 12 with the nucleotide marker 11 in an auto-device (i.e., feeder device, stirrer device or combination thereof) to form a nucleotide marker-contained material.

Still referring to FIGS. 1, 2 and 3, by way of example, the exogenous biomolecular tracing method in accordance with the preferred embodiment of the present invention includes the step S3: exogenously combining the nucleotide marker-contained material with the aquatic creature 21 or the aquatic product 22 in an auto-device (i.e., feed-supplying device, dipping device, painting device, sprayer device or combinations thereof) to form an exogenously-marked aquatic creature or an exogenously-marked aquatic product.

Still referring to FIGS. 1, 2 and 3, by way of example, exogenously combining the nucleotide marker-contained material with the aquatic creature 21 or the aquatic product 22 includes a feeding process, a dipping process, a painting process, a spraying process or combinations thereof. In another embodiment, by way of example, the nucleotide marker-contained material can be selectively formed as an oral vaccine or a vaccine product.

Still referring to FIGS. 1, 2 and 3, by way of example, the exogenous biomolecular tracing method in accordance with the preferred embodiment of the present invention includes the step S4: automatically, semi-automatically or manually identifying a DNA sample received from the exogenously-marked aquatic creature or the exogenously-marked aquatic product with at least one primer pair 30 in an exogenous biomolecular tracing procedure with the DNA testing device 3 to obtain an exogenously-marked identification result 4.

Still referring to FIGS. 1, 2 and 3, by way of example, the exogenous biomolecular tracing method in accordance with the preferred embodiment of the present invention includes: automatically, semi-automatically or manually transmitting the exogenously-marked identification result 4 to a device (e.g., computer display device, mobile communication device or other devices) for displaying a correct source of original aquatic creature or product or an incorrect source of original aquatic creature or product.

Still referring to FIGS. 1, 2 and 3, by way of example, the primer pair 30 is applied to execute a test of polymerase chain reaction (PCR) or loop-mediated isothermal amplification (LAMP). In another embodiment, the exogenously-marked identification result 4 includes a code (e.g., 101100 of LAMP reaction) which can be applied to identify the original aquatic creature or product.

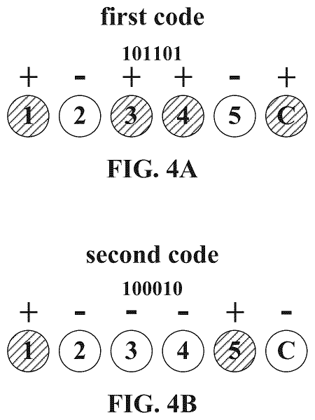

FIG. 4 shows a photograph view of a LAMP reaction applied in the exogenous biomolecular tracing system for aquatic creatures and products in accordance with a preferred embodiment of the present invention. Turning now to FIG. 4, by way of example, a predetermined number of test tubes (e.g., 4 tubes) of LAMP reaction is applied, with each test tube containing DNA samples, enzymes, buffer solutions and primer pairs, with the exogenously-marked identification result including positive (solution color changing to fluorescent green) identified as “+” and assigned code “1” and negative (remaining solution orange color) identified as “−” and code “0”.

FIG. 4A shows a schematic view of a first code of LAMP reaction generated in the exogenous biomolecular tracing system for aquatic creatures and products in accordance with a first embodiment of the present invention. Turning now to FIG. 4A, by way of example, the exogenous biomolecular tracing system generates a first 6-digit code (i.e., 101101) or more of LAMP reaction, with a sixth position of 6-digit code (i.e., control number) showing a positive result (labelled+) as passing a test.

FIG. 4B shows a schematic view of a second code of LAMP reaction generated in the exogenous biomolecular tracing system for aquatic creatures and products in accordance with a second embodiment of the present invention. Turning now to FIG. 4B, by way of example, the exogenous biomolecular tracing system generates a second 6-digit code (i.e., 100010) or more of LAMP reaction, with a sixth position of 6-digit code (i.e., control number) showing a negative result (labelled-) as failing a test.

Still referring to FIGS. 1, 2 and 3, by way of example, the nucleotide marker 11 is selected from a predetermined sequence, including Seq. 1 of Hermetia illucens, Seq. 2 of housefly, Seq. 3 of yeast fungus, Seq. 4 of Agaricus blazei, Seq. 5 of paddy rice, Seq. 6 of corn, Seq. 7 of soy bean, Seq. 8 of sweet potato, Seq. 9 of peanut, Seq. 10 of Bidens alba, Seq. 11 of tomato, Seq. 12 of wheat, Seq. 13 of banana, which can be identified by primer pairs, as best shown in Tables. 1-13.

TABLE 1
Primer pairs for identifying Seq. 1
of Hermetia illucens

| | | |
|---|---|---|
| F3 | 18 | TACTTCCCCCCTCTCTCA |
| B3 | 22 | CGATCAAATGTAATTCCTGTCG |
| FIP | 40 | CAATACCAGCGGCTAAGGGG TCTATAGTAGATGCCGGAGC |
| BIP | 42 | TTCCCTTCACTTAGCCGGGA ATCGCATATTGATTACTGTTGT |
| F2 | 20 | TCTATAGTAGATGCCGGAGC |
| F1c | 20 | CAATACCAGCGGCTAAGGGG |
| B2 | 22 | ATCGCATATTGATTACTGTTGT |
| B1c | 20 | TTCCCTTCACTTAGCCGGGA |

TABLE 2
Primer pairs for identifying Seq. 2 of housefly

| | | |
|---|---|---|
| F3 | 21 | GTTGAACTGTTTATCCACCTT |
| B3 | 19 | CAGCAAGAACAGGAAGAGA |
| FIP | 47 | AGAAATTCCTGCTAAGTGAAGAGAG |
| | | ATCATCAATTATTGCTCATGGT |
| BIP | 49 | TACGATCAACAGGAATTACATTCGA |
| | | TGCAGTAATTACAACTGATCAAAC |
| F2 | 22 | ATCATCAATTATTGCTCATGGT |
| F1c | 25 | AGAAATTCCTGCTAAGTGAAGAGAG |
| B2 | 24 | TGCAGTAATTACAACTGATCAAAC |
| B1c | 25 | TACGATCAACAGGAATTACATTCGA |

TABLE 3
Primer pairs for identifying Seq. 3 of
yeast fungus

| | | |
|---|---|---|
| F3 | 21 | TGAGTGATACTCTTTGGAGTT |
| B3 | 18 | GCCTAGACGCTCTCTTCT |
| FIP | 41 | AGCACGCAGAGAAACCTCTC AAATTGCTGGCCTTTTCATTG |
| BIP | 43 | AGGTATAATGCAAGTACGGTCGT |
| | | CGATAACGTTCCAATACGCT |
| F2 | 21 | AAATTGCTGGCCTTTTCATTG |
| F1c | 20 | AGCACGCAGAGAAACCTCTC |
| B2 | 20 | CGATAACGTTCCAATACGCT |
| B1c | 23 | AGGTATAATGCAAGTACGGTCGT |

TABLE 4
Primer pairs for identifying Seq. 4 of

| | Agaricus blazei | |
|---|---|---|
| F3 | 22 | TGTCACTCATTATACTCTGTCG |
| B3 | 18 | CCTCGGAATACCAAGGAG |
| FIP | 46 | GATCCGTTGCTGAAAGTTGTATTAC |
| | | ATTGAATGTCTTTACATGGGC |
| BIP | 43 | TTGGCTCTCGCATCGATGAAG |
| | | AAAGATTCGATGATTCACTGAA |
| F2 | 21 | ATTGAATGTCTTTACATGGGC |
| F1c | 25 | GATCCGTTGCTGAAAGTTGTATTAC |
| B2 | 22 | AAAGATTCGATGATTCACTGAA |
| B1c | 21 | TTGGCTCTCGCATCGATGAAG |

TABLE 5
Primer pairs for identifying Seq. 5 of paddy rice

| | | |
|---|---|---|
| F3 | 16 | CCGGGCCGCAACAGAA |
| B3 | 20 | CTCGATGGTTCACGGGATTC |
| FIP | 40 | AACTCGTGGTATCGCGCCGC TCAAGGAACACAGCGATACG |
| BIP | 42 | AATCCACACGACTCTCGGCAAC |
| | | TCGCATTTCGCTACGTTCTT |
| F2 | 20 | TCAAGGAACACAGCGATACG |
| F1c | 20 | AACTCGTGGTATCGCGCCGC |
| B2 | 20 | TCGCATTTCGCTACGTTCTT |
| B1c | 22 | AATCCACACGACTCTCGGCAAC |

TABLE 6
Primer pairs for identifying Seq. 6 of corn

| | | |
|---|---|---|
| F3 | 20 | GAAAGCCCGATCCAGCAATA |
| B3 | 18 | CCGATTCACCGCCTACGT |
| FIP | 41 | TGTCATGATCGCGCACTCGAC CGTGAGTGAAGAAGGGCAAT |
| BIP | 40 | CGAGGAAGAAGCCCCGGCTA GCCCAGTCATTCCGAAGAAC |
| F2 | 20 | CGTGAGTGAAGAAGGGCAAT |
| F1c | 21 | TGTCATGATCGCGCACTCGAC |
| B2 | 20 | GCCCAGTCATTCCGAAGAAC |
| B1c | 20 | CGAGGAAGAAGCCCCGGCTA |

TABLE 7
Primer pairs for identifying Seq. 7 of soy bean

| | | |
|---|---|---|
| F3 | 18 | GCCTCGTGGTTGGTTGAA |
| B3 | 20 | GCTTAAACTCAGCGGGTAGC |
| FIP | 38 | GATTGGTCTCGAGCGTGGCT TGGGTTCATGGCCGACTT |
| BIP | 38 | CGAGCCGGTCAGTTCTGGAC TGAGGTCTCGTTGGGAGC |
| F2 | 18 | TGGGTTCATGGCCGACTT |
| F1c | 20 | GATTGGTCTCGAGCGTGGCT |
| B2 | 18 | TGAGGTCTCGTTGGGAGC |
| B1c | 20 | CGAGCCGGTCAGTTCTGGAC |

TABLE 8
Primer pairs for identifying Seq. 8 of
sweet potato

| | | |
|---|---|---|
| F3 | 18 | CCTCGGGCGACTAACGAA |
| B3 | 20 | GCGTTCAAAGACTCGATGGT |
| FIP | 38 | CCGAGCGATCCGCAAAGACG CGCGCCAAGGAATATCGT |
| BIP | 41 | TCGGCAACGGATATCTCGGCT ACGGGATTCTGCAATTCACA |
| F2 | 18 | CGCGCCAAGGAATATCGT |
| F1c | 20 | CCGAGCGATCCGCAAAGACG |
| B2 | 20 | ACGGGATTCTGCAATTCACA |
| B1c | 21 | TCGGCAACGGATATCTCGGCT |

TABLE 9
Primer pairs for identifying Seq. 9 of peanut

| | | |
|---|---|---|
| F3 | 15 | CGCGGAAAGCGCCAA |
| B3 | 20 | GCGTTCAAAGACTCGATGGT |
| FIP | 39 | TGGTCACTCGTCGCCCCGA GGAAGCCAAACGTTTCTGCT |
| BIP | 41 | TCGGCAACGGATATCTCGGCT ACGGGATTCTGCAATTCACA |
| F2 | 20 | GGAAGCCAAACGTTTCTGCT |
| F1c | 19 | TGGTCACTCGTCGCCCCGA |
| B2 | 20 | ACGGGATTCTGCAATTCACA |
| B1c | 21 | TCGGCAACGGATATCTCGGCT |

TABLE 10
Primer pairs for identifying Seq. 10 of
Bidens alba

| | | |
|---|---|---|
| F3 | 21 | AACCAATTATCCAACCATTCC |
| B3 | 18 | CAGCCCAAATCGCCTTAC |
| FIP | 44 | CATTTGACCGCGTACCATTAAAG |
| | | CTTGACTTTCTGGGTTATCGT |
| BIP | 43 | GTATTGTTCCAATTATGCCTCTGGT |
| | | GATGCCCCAATGCGTTAC |
| F2 | 21 | CTTGACTTTCTGGGTTATCGT |
| F1c | 23 | CATTTGACCGCGTACCATTAAAG |
| B2 | 18 | GATGCCCCAATGCGTTAC |
| B1c | 25 | GTATTGTTCCAATTATGCCTCTGGT |

TABLE 11
Primer pairs for identifying Seq. 11 of tomato

| | | |
|---|---|---|
| F3 | 18 | GCCCAGCCTTAGAATCGG |
| B3 | 18 | CCCAAACAACCCGACTCG |
| FIP | 35 | TTCCAGGGGACTTGGGCCC GGCTCCGTCGTCCGAA |
| BIP | 38 | GCGCCAGAGAGGGTGAGAGC TAGACAGCGCCTCGTGGT |
| F2 | 16 | GGCTCCGTCGTCCGAA |
| F1c | 19 | TTCCAGGGGACTTGGGCCC |
| B2 | 18 | TAGACAGCGCCTCGTGGT |
| B1c | 20 | GCGCCAGAGAGGGTGAGAGC |

TABLE 12
Primer pairs for identifying Seq. 12 of wheat

| | | |
|---|---|---|
| F3 | 18 | AATGCCTCGACCACCTCC |
| B3 | 20 | TCGCATTTCGCTACGTTCTT |
| FIP | 40 | CCGGGTTAGGCACAGTGTTCC GGGCTCGGGGTAAAACAAC |
| BIP | 42 | GGCATGTCTAGCTTGCTAGCCG GATATCCGTTGCCGAGAGTC |
| F2 | 19 | GGGCTCGGGGTAAAACAAC |
| F1c | 21 | CCGGGTTAGGCACAGTGTTCC |
| B2 | 20 | GATATCCGTTGCCGAGAGTC |
| B1c | 22 | GGCATGTCTAGCTTGCTAGCCG |

TABLE 13
Primer pairs for identifying Seq. 13 of banana

| | | |
|---|---|---|
| F3 | 16 | GCTGCATGCAGGAGGC |
| B3 | 17 | TGCCCTTAGCCGGATGG |
| FIP | 41 | TCATCGATGCGAGAGCCGAGAT ATTCCGACGGTGACCCCAT |
| BIP | 42 | AGCGAAATGCGATACCTGGTGT CGCAACTTGCGTTCAAAGAC |
| F2 | 19 | ATTCCGACGGTGACCCCAT |
| F1c | 22 | TCATCGATGCGAGAGCCGAGAT |
| B2 | 20 | CGCAACTTGCGTTCAAAGAC |
| B1c | 22 | AGCGAAATGCGATACCTGGTGT |

Although the invention has been described in detail with reference to its presently preferred embodiment, it will be understood by one of ordinary skills in the art that various modifications can be made without departing from the spirit and the scope of the invention, as set forth in the appended claims.

What is claimed is:

1. An exogenous biomolecular tracing system comprising:
a nucleotide marker extracted from a marker source organism with an extraction device;
a nucleotide marker-contained material formed from combining a basic material with the nucleotide marker;
an exogenously-marked aquatic creature or an exogenously-marked aquatic product formed from exogenously combining the nucleotide marker-contained material with an aquatic creature or an aquatic product;
at least one primer pair corresponding to the nucleotide marker; and
an exogenously-marked identification result obtained by identifying a DNA sample received from the exogenously-marked aquatic creature or the exogenously-marked aquatic product with the at least one primer pair in an exogenous biomolecular tracing procedure with a DNA testing device;
wherein the exogenously-marked identification result shows a correct source of original aquatic creature or product or an incorrect source of original aquatic creature or product.

2. The system as defined in claim 1, wherein the nucleotide marker is selected from an insect extract, a plant extract, a microorganism extract or combinations thereof.

3. The system as defined in claim 1, wherein the basic material is selected from a solution, a powder, a fragment, a solid or combinations thereof.

4. The system as defined in claim 1, wherein the aquatic creature or the aquatic product is selected from fishes, shrimps, shellfishes, algae or combinations thereof.

5. The system as defined in claim 1, wherein the aquatic creature or the aquatic product is selected from edible aquatic creatures, ornamental aquatic creatures, live aquatic creatures, frozen aquatic creatures, aquatic fry or aquatic baits.

6. An exogenous biomolecular tracing method comprising:
extracting a nucleotide marker from a marker source organism;
combining a basic material with the nucleotide marker to form a nucleotide marker-contained material;
exogenously combining the nucleotide marker-contained material with an aquatic creature or an aquatic product to form an exogenously-marked aquatic creature or an exogenously-marked aquatic product; and
identifying a DNA sample received from the exogenously-marked aquatic creature or the exogenously-marked aquatic product with at least one primer pair in an exogenous biomolecular tracing procedure to obtain an exogenously-marked identification result.

7. The method as defined in claim 6, wherein the nucleotide marker is selected from a DNA extract, a DNA extract liquid, a DNA extract powder, a DNA extract-related material or combinations thereof.

8. The method as defined in claim 6, wherein exogenously combining the nucleotide marker-contained material with the aquatic creature or the aquatic product includes a feeding process, a dipping process, a painting process, a spraying process or combinations thereof.

9. The method as defined in claim 6, wherein the at least one primer pair is applied to execute a test of polymerase chain reaction (PCR) or loop-mediated isothermal amplification (LAMP).

10. The method as defined in claim 6, wherein the exogenously-marked identification result includes a combination set of codes or code forms for tracing the exogenously-marked aquatic creature or the exogenously-marked aquatic product.

11. An exogenous biomolecular tracing method comprising:
extracting a first nucleotide marker from a first marker source organism;
combining a first basic material with the first nucleotide marker to form a first nucleotide marker-contained material;
extracting a second nucleotide marker from a second marker source organism;
combining a second basic material with the second nucleotide marker to form a second nucleotide marker-contained material;
exogenously combining the first nucleotide marker-contained material with an aquatic creature or an aquatic product to form a first exogenously-marked aquatic creature or a first exogenously-marked aquatic product in a first stage;
exogenously combining the second nucleotide marker-contained material with the first exogenously-marked aquatic creature or the first exogenously-marked aquatic product to form a second exogenously-marked aquatic creature or a second exogenously-marked aquatic product in a second stage; and
identifying a DNA sample received from the second exogenously-marked aquatic creature or the second exogenously-marked aquatic product with at least one primer pair in an exogenous biomolecular tracing procedure to obtain an exogenously-marked identification result.

12. The method as defined in claim 11, wherein the first or second nucleotide marker is selected from a DNA extract, a DNA extract liquid, a DNA extract powder, a DNA extract-related material or combinations thereof.

13. The method as defined in claim 11, wherein exogenously combining the first or second nucleotide marker-contained material with the aquatic creature or the aquatic product includes a feeding process, a dipping process, a painting process, a spraying process or combinations thereof.

14. The method as defined in claim 11, wherein the at least one primer pair is applied to execute a test of polymerase chain reaction (PCR) or loop-mediated isothermal amplification (LAMP).

15. The method as defined in claim 11, wherein the exogenously-marked identification result includes a combination set of codes or code forms for tracing the second exogenously-marked aquatic creature or the second exogenously-marked aquatic product.


SEQUENCE LISTING (117 sequences)

SEQ ID NO: 1 - DNA, 685 bp, Unknown
  Hermetia illucens
aaagatattggtacattatattttatctttggagcctgagctggtatagtgggaacttctttaagaattcttattcgagccgaattaggtcatccaggagctttaattggagatgaccaaatttataacgtaattgttactgcccatgcttttgtaataattttctttatagtaatgccaattataattggtggatttggtaattgacttgttcccctaatattaggagcccctgatatagctttcccacgaataaataacataagtttctggttacttcccccctctctcactcttttattagcctcttctatagtagatgccggagcaggaaccggttgaactgtctacccccccttagccgctggtattgctcatagtggagcttcagttgacctagctattttttcccttcacttagccgggatttcttcaattttaggggctgtaaattttattacaacagtaatcaatatgcgatcgacaggaattacatttgatcgaatacctctatttgtttgatcagtagtaattacagcagtacttctccttctgtcacttcctgttcttgctggagctattaccatattattaacagatcgaaatttaaatacttctttcttcgacccagccggaggaggagatcccattctttatcaacatttattttgattttttggtcacc

SEQ ID NO: 2 - DNA, 658 bp, Unknown
  Housefly
tactttatattttatcttcggagcatgatctggtatagtaggaacatcattaagaattttaattcgagctgaattaggacaccctggtgctctaattggagacgatcaaatttataatgttattgtaacagctcatgcttttattataattttctttatagtaatacctattataattggagggtttggaaattgattagttcctttaatattaggagctccagatatagcattccctcgaatgaataatataagtttttgattattacctcctgcattaactctattattagtaagaagtatagtagaaaagggagctggaacaggttgaactgtttatccacctttatcatcaattattgctcatggtggagcttcagttgatttagctattttctctcttcacttagcaggaatttcttcaattttaggagcagtaaattttattacaactgttattaacatacgatcaacaggaattacattcgatcgaatgcctttatttgtttgatcagttgtaattactgcattattattattattatctcttcctgttcttgctggagctattactatactattaactgatcgaaatttaaatacttcattctttgacccagctggaggaggtgatccaattctttaccaacacttattc

SEQ ID NO: 3 - DNA, 796 bp, Unknown
  Yeast
agagagcggaaggatcattaaagaaatttaataattttgaaaatggattttttttgttttggcaagagcatgagagcttttactgggcaagaagacaagagatggagagtccagccgggcctgcgcttaagtgcgcggtcttgctaggcttgtaagtttctttcttgctattccaaacggtgagagatttctgtgcttttgttataggacaattaaaaccgtttcaatacaacacactgtggagttttcatatctttgcaactttttctttgggcattcgagcaatcggggcccagaggtaacaaacacaaacaattttatttattcattaaatttttgtcaaaaacaagaattttcgtaactggaaattttaaaatattaaaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttccttctcaaacattctgtttggtagtgagtgatactctttggagttaacttgaaattgctggccttttcattggatgttttttttttccaaagagaggtttctctgcgtgcttgaggtataatgcaagtacggtcgttttaggttttaccaactgcggctaatcttttttatactgagcgtattggaacgttatcgataagaagagagcgtctaggcgaacaatgttcttaaagttgacctcaaatcaggtagaac

SEQ ID NO: 4 - DNA, 773 bp, Unknown
  Agaricus blazei
ggaagtaaaagtcgtaacaaggtttccgtaggtgaacctgcggaaggatcattattgaattatgtttctagataggttgtagctggctcttttagagcatgtgcacgcctgtttggacttcattttcatccacctgtgcacctattgtagtctttggttgggttaggaggaagtgatcattgtatcagcatctgctgggagtgaggacttgcattgtgaaagctttgctgtccttgatgtgatcatggaatctttttctcactagagtctatgtcactcattatactctgtcgaatgtcattgaatgtctttacatgggcttgtatgcctatgaaaattgtaatacaactttcagcaacggatctcttggctctcgcatcgatgaagaacgcagcgaaatgcgataagtaatgtgaattgcagaattcagtgaatcatcgaatctttgaacgcatcttgcgctccttggtattccgaggagcatgcctgtttgagtgtcattaaattctcaactctcttatacttttttgtaaaagagagcttggactgtggaggcttgctggccactttttggggtcagctcctctgaaatgcattagcggaaccgtttgcgatctgccacaagtgtgataagttatctacactggcgaggggattgctctctgtaatgttcagcttctaattgtctctactttgtgagactacttttgaatgcttgacctcaaatcaggtaggactacccgctgaacttaagcatatcaataagcggagga

SEQ ID NO: 5 - DNA, 1155 bp, Unknown
  Rice
aggtgaacctgcggaaggatcattgtcgtgaccctgaccaaaacagaccgcgaacgcgtcacccctgcccgccgagcgctcgcgcgcgaggcaaccgaggcccccgggccgcaacagaacccacggcgccgacggcgtcaaggaacacagcgatacgccccgcgccggcccggtcggccctggccgtccggcggcgcggcgcgataccacgagttaaatccacacgactctcggcaacggatatctcggctctcgcatcgatgaagaacgtagcgaaatgcgatacctggtgtgaattgcagaatcccgtgaaccatcgagtctttgaacgcaagttgcgcccgaggccatccggccgagggcacgcctgcctgggcgtcacgccaaaagacgctccgcgcgccccccctatccgggagggcgcggggacgcggtgtctggccccccgcgcctcgcggcgcggtgggccgaagctcgggctgccggcgaagcgtgccgggcacagcgcatggtggacagctcacgctggctctaggccgcagtgcaccccggcgcgcggccggcgcggtggcccctcaggacccaaacgcaccgagagcgaacgcctcggaccgcgaccccaggtcaggcgggactacccgctgagtttaagcatataaataagcggaggagaagaaacttacgaggattcccctagtaacggcgagcgaaccgggagatgcccagcttgagaatcgggcggccgcgccgtccgaattgtagtctggagaggcgtcctcagcgacggaccgggcccaagtcccctggaaaggggcgcctgggagggtgagagccccgtccggcccggaccctgtcgccccacgaggcgccgtcaacgagtcgggttgtttgggaatgcagcccaaatcgggcggtaaactccgtccaaggctaaatacaggcgagagaccgatagcgaacaagtaccgcgagggaaagatgaaaaggactttgaaaagagagtcaaagagtgcttgaaattgccgggagggaagcggatgggggccggcgatgcgccccggccgtatgcggaacggcttcggctggtccgccgatcggctcggggcgtggactgttgtcggccgcgccggcggccaaagcccgggggctccgcgc

SEQ ID NO: 6 - DNA, 840 bp, Unknown
  Corn
atcatagtcaaaatctgagtttgatcctggctcagaaggaacgctagctatatgcttaacacatgcaagtcgaacgttgttttcggggagctgggcagaaggaaaagaggctcctagctaaagttgtctcgccctgcctgcagggcgcgcgcaacggctttgacctaacggcctccgtttgctggaatcggaatagttgagaacaaagtggcgaacgggtgcgtaacgcgtgggaatctgccgaacagttcgggccaaatcctgaagaaagctcaaaagcgctgtttgatgagcctgcgtagtattaggtagttggtcaggtaaaggctgaccaagccaatgatgcttagctggtcttttcggatgatcagccacactgggactgagacacggcccggactcccacggggggcagcagtggggaatcttggacaatgggcgaaagcccgatccagcaatatcgcgtgagtgaagaagggcaatgccgcttgtaaagctctttcgtcgagtgcgcgatcatgacaggactcgaggaagaagccccggctaactccgtgccagcagccgcggtaagacggggggggcaagtgttcttcggaatgactgggcgtaaagggcacgtaggcggtgaatcgggttgaaagtgaaagtcgccaaaaagtggcggaatgctctcgaaaccaattcacttgagtgagacagaggagagtggaatttcgtgtgtaggggtgaaatccgtagatctacgaaggaacgccaaaagcgaaggcagctctctgggtccctaccgacgctggggtgcgaaagcatggggagcgaacaggattagataccctggta

SEQ ID NO: 7 - DNA, 700 bp, Unknown
  Soy bean
cgtaacaaggtttccgtaggtgaacctgcggaaggatcattgtcgatgcctcacaatcagattgacccgcgaacttgtttattcatctaccgtcgggagggaggggatgaccacggcgccccgtgcgcccggcctcctcgtcctcgcgacaaacacaaaccccggcgcttcgtgcgccaaggaactcaaatctgttaagtgcgactcccgggggcccggagacggtgtcccgcgggagtcgtcacgacacaacatttacatacaatgactctcggcaacggatatctcggctcttgcatcgatgaagaacgtagcgaaatgcgatacttggtgtgaattgcagaatcccgtgaaccatcgagtctttgaacgcaagttgcgcccgaagccattaggccgagggcacgcctgcctgggtgtcacacatcgtttccccaacgcaaacatgtaacaatgttgctgcgcggggtgtatgctgacctcccgcgagcacccgcctcgtggttggttgaaatctgggttcatggccgacttcgccgtgataaaatggtggatgagccacgctcgagaccaatcacgtgcgagccggtcagttctggacccatcgacgaccctttgcgtgcacgcacgctcccaacgagacctcaggtcaggcggggctacccgctgagtttaagcatatcaataagcggaggaaaag

SEQ ID NO: 8 - DNA, 770 bp, Unknown
  Sweet potato
gaggcaataacaggtctgtgatgcccttagatgttctgggccgcacgcgcgctacactgatgtattcaacgagcatatagccttggccgacaggcccgggtaatctttgaaatttcatcgtgatggggatagatcattgcaattgttggtcttcaacgaggaattcctagtaagcgcgagtcatcagctcgcgttgactacgtccctgccctttgtacacaccgcccgtcgctcctaccgattgaatgatccggtgaagtgttcggatcgcggcgacgtgggcggttcgctgcccgcgacgtcgcgagaagtccactgaaccttatcatttagaggaaggagaagtcgtaacaaggtttccgtaggtgaacctgcggaaggatcattgtcgaaacctgcacagcagaacgaccagagaacgcgtttgttattcaatctcctcgtccgggcgcatgcctcgggcgactaacgaacccccggcgcggaacgcgccaaggaatatcgtactgagatggccagccgcccgtgccccgtctttgcggatcgctcgggaggcgtcggcgtcttacttaataaaatacgactctcggcaacggatatctcggctctcgcatcgatgaagaacgtagcgaaatgcgatacttggtgtgaattgcagaatcccgtgaaccatcgagtctttgaacgcaagttgcgcccgaagccgtcaggccgagggcacgtctgcctgggcgtcacgcatcgcgtcgccccccgctcgatcctcggtc

SEQ ID NO: 9 - DNA, 660 bp, Unknown
  Peanut
tttccgtaggtgaacctgcggaaggatcattgtcgatgccgcacaaaccaggattgacgcgcgaacgagtccacaaacacccgaggcggggaagggccggccgtgcgcggccggcgccccgtctcaaacaagaacaaaaccccggcgcggaaagcgccaaggaagccaaacgtttctgctctccccgccggctccggagacggcatccggtcggggcgacgagtgaccacaagagttaagaacgactctcggcaacggatatctcggctcttgcatcgatgaagaacgtagcgaaatgcgatacttggtgtgaattgcagaatcccgtgaaccatcgagtctttgaacgcaagttgcgcccgaagcccttaggctgagggcacgcctgcctgggtgtcaccaaaaggcgcccccccgtctcgcccgtcccagggcacggggagggggcgaacgttggcctcccgggagcccctggctcgcggttggttcaaagagacgggctcttggtggggagcggcaccgcggcagatggtggtcgagaacaaccctcgtggccagtcgcgcgcgcctctcccccggttcaaggcacggcgacccgcgggcgacgtggatcgtcccgagcgcgacctcaggtcaggcggggctacccgctgagtttaa

SEQ ID NO: 10 - DNA, 751 bp, Unknown
  Bidens alba
aaagatgcttcctctttgcatttattaagattctttctccatgagtgtcataattgggatagtattattacttcaaattcaaagaaagccagttcttctttttcaaaaagaaatcacagactattcttcttcctatatacttcttatgtatgtgaatatgaatctagcttcctctttctccgtaaccaatcttctcacttacgatcaacatcttctggagcccttattgaacgaatatatttctatggaaaaatagagcatcttgcagaagtctttgccagggcttttcaagctaatttatggttgttcaaagattctttcatgcattatgttaggtatcaaggaaaatcaattcttgcttcaaaggggacgtttcttttgatgactaaatggaaatattactttttcaatttctggaaatcttatttttacctgtggtctcaaccaggaaggatttatataaaaataaaccaattatccaaccattcccttgactttctgggttatcgttcaagtgtgcggctaaagcctttaatggtacgcggtcaaatgctagaaaatacatttttaatcgataatgctattaagaagtttgatagtattgttccaattatgcctctggttggatcattggctaaatcgaaattttgtaacgcattggggcatcctattggtaaggcgatttgggctgatttctcagattctgatattattgagcgctttggccgtatatacagaaatctttctcatt

SEQ ID NO: 11 - DNA, 766 bp, Unknown
  Potato
ccgagcgcgcggctggcctaaatgcgagtccacgtcgacggacgtcgcggcaagtggtggttgaaactcaactctctcttgttgtcgcggctacagcccgtcgtgcgtccggactcccagaccctgattgcgcctaggcgctccgaccgcgaccccaggtcaggcgggattacccgctgagtttaagcatatcaataagcggaggaaaagaaacttacaaggattcccttagtaacggcgagcgaaccgggaacagcccagccttagaatcgggcggctccgtcgtccgaattgtagtctggagaagcgtcctcagcggcggaccgggcccaagtcccctggaagggggcgccagagagggtgagagccccgttgtgcccggaccctgtcgcaccacgaggcgctgtctacgagtcgggttgtttgggaatgcagcccaaatcgggcggtgaattccgtccaaggctaaatacgggcgagagaccgatagcgaacaagtaccgcgagggaaagatgaaaaggactttgaaaagagagtcaaagagtgcttgaaattgtcgggagggaagcggatgggggccggcgatgcgccccggtcggatgtggaacggtgacgagccggtccgccgatcgactcggggcgtggaccagcgtggattgggggggcggccaaagcccgggctctcgatacgcccgcggaacgccgtctcctcgattgtggtaggcagcgcgcgcctctggcgtgcttcggcatctgcgcgctccg

SEQ ID NO: 12 - DNA, 703 bp, Unknown
  Wheat
cgtaacaaggtttccgtaggtgaacctgcggaaggatcattgtcgtgaccctgaccaaaacagactgcgcacgcgtcatccaatccgtcggcgacggcaccgtccgtcgctcggccaatgcctcgaccacctcccctcctcggagcgggtgggggctcggggtaaaacaacccacggcgccgaaggcgtcaaggaacactgtgcctaacccgggggcatgtctagcttgctagccgtccctcgtgttgcaaatctatttaatccacacgactctcggcaacggatatctcggctctcgcatcgatgaagaacgtagcgaaatgcgatacctggtgtgaattgcagaatcccgcgaaccatcgagtctttgaacgcaagttgcgcccgaggccactcggccgagggcacgcctgcctgggcgtcacgccaaaacacgctcccaaccactctcaacgggaatcgggatgcggcatctggtccctcgtctctcaagggacggtggaccgaagattgggctgccggcgtaccgcgccggacacagcgcatggtgggcgtcctcgctttatcaatgcagtgcatccggcgcgcagctggcattatgacctttgaacgacccaacaaacgaagcgcacgtcgcttcgaccgcgaccccaggtcaggcgggactacccgctgagtttaagcatatcaataagcggaaaag

SEQ ID NO: 13 - DNA, 678 bp, Unknown
  Banana
tccgtaggtgaacctgcggaaggatcattgtcgagacccactgacgaggacgaccgtgaatgcgtcaacgattgctcgtcgggctcgtcccgacaacacccccgaatgtcggtccgccctcgggcgggacgaccgaggggatgaactaccaaccccggcgcggatagcgccaaggaacacgaacatcgaagtcggagggcctcgctgcatgcaggaggctacaattccgacggtgaccccattggacgactctcggcaacggatatctcggctctcgcatcgatgaagaacgtagcgaaatgcgatacctggtgtgaattgcagaatcccgtgaaccatcgagtctttgaacgcaagttgcgcccgaggccatccggctaagggcacgcctgcctgggcgtcacgctttcgacgcttcgtcgttgccccctcggggggtgggggcgaacgtggaggatggccccccgtgccggaaaggtgcggttggccgaagagcgggccgtcggtggttgtcgaacacgacgcgtggtggatgccttgtgcgagccgtacgtcgtgccttcgggacccgggcgaggcctcgaggacccaagtcgtggtgcgagtcgatgccacggaccgcgaccccaggtcaggtggggctacccgctgagtttaagcatatcaataagcggagga

SEQ ID NO: 14 - DNA, 18 bp, artificial sequence
  primer
tacttcccccctctctca

SEQ ID NO: 15 - DNA, 22 bp, artificial sequence
  primer
cgatcaaatgtaattcctgtcg

SEQ ID NO: 16 - DNA, 40 bp, artificial sequence
  primer
caataccagcggctaaggggtctatagtagatgccggagc

SEQ ID NO: 17 - DNA, 42 bp, artificial sequence
  primer
ttcccttcacttagccgggaatcgcatattgattactgttgt

SEQ ID NO: 18 - DNA, 20 bp, artificial sequence
  primer
tctatagtagatgccggagc

SEQ ID NO: 19 - DNA, 20 bp, artificial sequence
  primer
caataccagcggctaagggg

SEQ ID NO: 20 - DNA, 22 bp, artificial sequence
  primer
atcgcatattgattactgttgt

SEQ ID NO: 21 - DNA, 20 bp, artificial sequence
  primer
ttcccttcacttagccggga

SEQ ID NO: 22 - DNA, 21 bp, artificial sequence
  primer
gttgaactgtttatccacctt

SEQ ID NO: 23 - DNA, 19 bp, artificial sequence
  primer
cagcaagaacaggaagaga

SEQ ID NO: 24 - DNA, 47 bp, artificial sequence
  primer
agaaattcctgctaagtgaagagagatcatcaattattgctcatggt

SEQ ID NO: 25 - DNA, 49 bp, artificial sequence
  primer
tacgatcaacaggaattacattcgatgcagtaattacaactgatcaaac

SEQ ID NO: 26 - DNA, 22 bp, artificial sequence
  primer
atcatcaattattgctcatggt

SEQ ID NO: 27 - DNA, 25 bp, artificial sequence
  primer
agaaattcctgctaagtgaagagag

SEQ ID NO: 28 - DNA, 24 bp, artificial sequence
  primer
tgcagtaattacaactgatcaaac

SEQ ID NO: 29 - DNA, 25 bp, artificial sequence
  primer
tacgatcaacaggaattacattcga

SEQ ID NO: 30 - DNA, 21 bp, artificial sequence
  primer
tgagtgatactctttggagtt

SEQ ID NO: 31 - DNA, 18 bp, artificial sequence
  primer
gcctagacgctctcttct

SEQ ID NO: 32 - DNA, 41 bp, artificial sequence
  primer
agcacgcagagaaacctctcaaattgctggccttttcattg

SEQ ID NO: 33 - DNA, 43 bp, artificial sequence
  primer
aggtataatgcaagtacggtcgtcgataacgttccaatacgct

SEQ ID NO: 34 - DNA, 21 bp, artificial sequence
  primer
aaattgctggccttttcattg

SEQ ID NO: 35 - DNA, 20 bp, artificial sequence
  primer
agcacgcagagaaacctctc

SEQ ID NO: 36 - DNA, 20 bp, artificial sequence
  primer
cgataacgttccaatacgct

SEQ ID NO: 37 - DNA, 23 bp, artificial sequence
  primer
aggtataatgcaagtacggtcgt

SEQ ID NO: 38 - DNA, 22 bp, artificial sequence
  primer
tgtcactcattatactctgtcg

SEQ ID NO: 39 - DNA, 18 bp, artificial sequence
  primer
cctcggaataccaaggag

SEQ ID NO: 40 - DNA, 46 bp, artificial sequence
  primer
gatccgttgctgaaagttgtattacattgaatgtctttacatgggc

SEQ ID NO: 41 - DNA, 43 bp, artificial sequence
  primer
ttggctctcgcatcgatgaagaaagattcgatgattcactgaa

SEQ ID NO: 42 - DNA, 21 bp, artificial sequence
  primer
attgaatgtctttacatgggc

SEQ ID NO: 43 - DNA, 25 bp, artificial sequence
  primer
gatccgttgctgaaagttgtattac

SEQ ID NO: 44 - DNA, 22 bp, artificial sequence
  primer
aaagattcgatgattcactgaa

SEQ ID NO: 45 - DNA, 21 bp, artificial sequence
  primer
ttggctctcgcatcgatgaag

SEQ ID NO: 46 - DNA, 16 bp, artificial sequence
  primer
ccgggccgcaacagaa

SEQ ID NO: 47 - DNA, 20 bp, artificial sequence
  primer
ctcgatggttcacgggattc

SEQ ID NO: 48 - DNA, 40 bp, artificial sequence
  primer
aactcgtggtatcgcgccgctcaaggaacacagcgatacg

SEQ ID NO: 49 - DNA, 42 bp, artificial sequence
  primer
aatccacacgactctcggcaactcgcatttcgctacgttctt

SEQ ID NO: 50 - DNA, 20 bp, artificial sequence
  primer
tcaaggaacacagcgatacg

SEQ ID NO: 51 - DNA, 20 bp, artificial sequence
  primer
aactcgtggtatcgcgccgc

SEQ ID NO: 52 - DNA, 20 bp, artificial sequence
  primer
tcgcatttcgctacgttctt

SEQ ID NO: 53 - DNA, 22 bp, artificial sequence
  primer
aatccacacgactctcggcaac

SEQ ID NO: 54 - DNA, 20 bp, artificial sequence
  primer
gaaagcccgatccagcaata

SEQ ID NO: 55 - DNA, 18 bp, artificial sequence
  primer
ccgattcaccgcctacgt

SEQ ID NO: 56 - DNA, 41 bp, artificial sequence
  primer
tgtcatgatcgcgcactcgaccgtgagtgaagaagggcaat

SEQ ID NO: 57 - DNA, 40 bp, artificial sequence
  primer
cgaggaagaagccccggctagcccagtcattccgaagaac

SEQ ID NO: 58 - DNA, 20 bp, artificial sequence
  primer
cgtgagtgaagaagggcaat

SEQ ID NO: 59 - DNA, 21 bp, artificial sequence
  primer
tgtcatgatcgcgcactcgac

SEQ ID NO: 60 - DNA, 20 bp, artificial sequence
  primer
gcccagtcattccgaagaac

SEQ ID NO: 61 - DNA, 20 bp, artificial sequence
  primer
cgaggaagaagccccggcta

SEQ ID NO: 62 - DNA, 18 bp, artificial sequence
  primer
gcctcgtggttggttgaa

SEQ ID NO: 63 - DNA, 20 bp, artificial sequence
  primer
gcttaaactcagcgggtagc

SEQ ID NO: 64 - DNA, 38 bp, artificial sequence
  primer
gattggtctcgagcgtggcttgggttcatggccgactt

SEQ ID NO: 65 - DNA, 38 bp, artificial sequence
  primer
cgagccggtcagttctggactgaggtctcgttgggagc

SEQ ID NO: 66 - DNA, 18 bp, artificial sequence
  primer
tgggttcatggccgactt

SEQ ID NO: 67 - DNA, 20 bp, artificial sequence
  primer
gattggtctcgagcgtggct

SEQ ID NO: 68 - DNA, 18 bp, artificial sequence
  primer
tgaggtctcgttgggagc

SEQ ID NO: 69 - DNA, 20 bp, artificial sequence
  primer
cgagccggtcagttctggac

SEQ ID NO: 70 - DNA, 18 bp, artificial sequence
  primer
cctcgggcgactaacgaa

SEQ ID NO: 71 - DNA, 20 bp, artificial sequence
  primer
gcgttcaaagactcgatggt

SEQ ID NO: 72 - DNA, 38 bp, artificial sequence
  primer
ccgagcgatccgcaaagacgcgcgccaaggaatatcgt

SEQ ID NO: 73 - DNA, 41 bp, artificial sequence
  primer
tcggcaacggatatctcggctacgggattctgcaattcaca

SEQ ID NO: 74 - DNA, 18 bp, artificial sequence
  primer
cgcgccaaggaatatcgt

SEQ ID NO: 75 - DNA, 20 bp, artificial sequence
  primer
ccgagcgatccgcaaagacg

SEQ ID NO: 76 - DNA, 20 bp, artificial sequence
  primer
acgggattctgcaattcaca

SEQ ID NO: 77 - DNA, 21 bp, artificial sequence
  primer
tcggcaacggatatctcggct

SEQ ID NO: 78 - DNA, 15 bp, artificial sequence
  primer
cgcggaaagcgccaa

SEQ ID NO: 79 - DNA, 20 bp, artificial sequence
  primer
gcgttcaaagactcgatggt

SEQ ID NO: 80 - DNA, 39 bp, artificial sequence
  primer
tggtcactcgtcgccccgaggaagccaaacgtttctgct

SEQ ID NO: 81 - DNA, 41 bp, artificial sequence; identical to SEQ ID NO: 73
  primer

SEQ ID NO: 82 - DNA, 20 bp, artificial sequence
  primer
ggaagccaaacgtttctgct

SEQ ID NO: 83 - DNA, 19 bp, artificial sequence
  primer
tggtcactcgtcgccccga

SEQ ID NO: 84 - DNA, 20 bp, artificial sequence
  primer
acgggattctgcaattcaca

SEQ ID NO: 85 - DNA, 21 bp, artificial sequence
  primer
tcggcaacggatatctcggct

SEQ ID NO: 86 - DNA, 21 bp, artificial sequence
  primer
aaccaattatccaaccattcc

SEQ ID NO: 87 - DNA, 18 bp, artificial sequence
  primer
cagcccaaatcgccttac

SEQ ID NO: 88 - DNA, 44 bp, artificial sequence
  primer
catttgaccgcgtaccattaaagcttgactttctgggttatcgt

SEQ ID NO: 89 - DNA, 43 bp, artificial sequence
  primer
gtattgttccaattatgcctctggtgatgccccaatgcgttac

SEQ ID NO: 90 - DNA, 21 bp, artificial sequence
  primer
cttgactttctgggttatcgt

SEQ ID NO: 91 - DNA, 23 bp, artificial sequence
  primer
catttgaccgcgtaccattaaag

SEQ ID NO: 92 - DNA, 18 bp, artificial sequence
  primer
gatgccccaatgcgttac

SEQ ID NO: 93 - DNA, 25 bp, artificial sequence
  primer
gtattgttccaattatgcctctggt

SEQ ID NO: 94 - DNA, 18 bp, artificial sequence
  primer
gcccagccttagaatcgg

SEQ ID NO: 95 - DNA, 18 bp, artificial sequence
  primer
cccaaacaacccgactcg

SEQ ID NO: 96 - DNA, 35 bp, artificial sequence
  primer
ttccaggggacttgggcccggctccgtcgtccgaa

SEQ ID NO: 97 - DNA, 38 bp, artificial sequence
  primer
gcgccagagagggtgagagctagacagcgcctcgtggt

SEQ ID NO: 98 - DNA, 16 bp, artificial sequence
  primer
ggctccgtcgtccgaa

SEQ ID NO: 99 - DNA, 19 bp, artificial sequence
  primer
ttccaggggacttgggccc

SEQ ID NO: 100 - DNA, 18 bp, artificial sequence
  primer
tagacagcgcctcgtggt

SEQ ID NO: 101 - DNA, 20 bp, artificial sequence
  primer
gcgccagagagggtgagagc

SEQ ID NO: 102 - DNA, 18 bp, artificial sequence
  primer
aatgcctcgaccacctcc

SEQ ID NO: 103 - DNA, 20 bp, artificial sequence
  primer
tcgcatttcgctacgttctt

SEQ ID NO: 104 - DNA, 40 bp, artificial sequence
  primer
ccgggttaggcacagtgttccgggctcggggtaaaacaac

SEQ ID NO: 105 - DNA, 42 bp, artificial sequence
  primer
ggcatgtctagcttgctagccggatatccgttgccgagagtc

SEQ ID NO: 106 - DNA, 19 bp, artificial sequence
  primer
gggctcggggtaaaacaac

SEQ ID NO: 107 - DNA, 21 bp, artificial sequence
  primer
ccgggttaggcacagtgttcc

SEQ ID NO: 108 - DNA, 20 bp, artificial sequence
  primer
gatatccgttgccgagagtc

SEQ ID NO: 109 - DNA, 22 bp, artificial sequence
  primer
ggcatgtctagcttgctagccg

SEQ ID NO: 110 - DNA, 16 bp, artificial sequence
  primer
gctgcatgcaggaggc

SEQ ID NO: 111 - DNA, 17 bp, artificial sequence
  primer
tgcccttagccggatgg

SEQ ID NO: 112 - DNA, 41 bp, artificial sequence
  primer
tcatcgatgcgagagccgagatattccgacggtgaccccat

SEQ ID NO: 113 - DNA, 42 bp, artificial sequence
  primer
agcgaaatgcgatacctggtgtcgcaacttgcgttcaaagac

SEQ ID NO: 114 - DNA, 19 bp, artificial sequence
  primer
attccgacggtgaccccat

SEQ ID NO: 115 - DNA, 22 bp, artificial sequence
  primer
tcatcgatgcgagagccgagat

SEQ ID NO: 116 - DNA, 20 bp, artificial sequence
  primer
cgcaacttgcgttcaaagac

SEQ ID NO: 117 - DNA, 22 bp, artificial sequence
  primer
agcgaaatgcgatacctggtgt